(19) United States
(12) Patent Application Publication
(10) Pub. No.: US 2026/0284003 A1
(43) Pub. Date: Sep. 24, 2026

(54) COMPOSITIONS AND METHOD FOR MANAGEMENT OF PAIN AND ALLERGY

(71) Applicants: ANJU MAJEED, Bangalore (IN); SHAHEEN MAJEED, Elk Ridge, UT (US); LAKSHMI MUNDKUR, Bangalore (IN); MOHAMMAD MANSOOR ANSARI, Bangalore (IN)

(72) Inventors: ANJU MAJEED, Bangalore (IN); SHAHEEN MAJEED, Elk Ridge, UT (US); LAKSHMI MUNDKUR, Bangalore (IN); MOHAMMAD MANSOOR ANSARI, Bangalore (IN)

(21) Appl. No.: 19/473,374

(22) PCT Filed: Apr. 12, 2024
(86) PCT No.: PCT/US2024/024233

§ 371 Date: Oct. 7, 2025

(30) Foreign Application Priority Data
Apr. 12, 2023 (IN) 202341026964

(51) Int. Cl. A61K 31/36; A61K 31/015; A61K 31/164; A61P 25/00; A61P 25/02; A61P 29/00; A61P 37/08

(52) U.S. Cl. CPC A61K 31/36; A61K 31/015; A61K 31/164; A61P 25/00; A61P 25/02; A61P 29/00; A61P 37/08

(57) ABSTRACT

The invention discloses a composition and method for therapeutically managing pain and allergy. The invention covers a composition comprising 4-10% Guineesine, β-Caryophyllene Palmitoylethanolamide, and pharmaceutically or nutraceutically acceptable excipients, which can be effectively formulated as tablets and capsules. The invention also covers use of the composition for effectively managing pain and allergy.

### CROSS-REFERENCE TO RELATED APPLICATION

This application in a US national phase application of PCT application no. PCT/US2024/024233, filed on 12 Apr. 2024, which in turn claims priority from Indian Provisional patent application no. IN202341026964, filed on 12 Apr. 2023, the contents of which are incorporated herein by reference

### FIELD OF INVENTION

The invention is directed towards a composition comprising 4-10% w/w Guineesine, β-Caryophyllene, and Palmitoylethanolamide. Further it also covers the use of the said composition in treating pain and allergy.

### BACKGROUND

Mast cells, part of innate and adaptive immune system, are omnipresent in the body and are found near externally exposed surfaces such as the epithelial lining of the skin, mucosa of the gastrointestinal tract, meningeal membrane of the CNS, and airways of the lung. It is this proximity to the external surface that induces immune response to the environmental stimuli, such as allergens and pathogens. It is the extreme sensitivity of mast cells that lead to pathological conditions, allergy, and anaphylaxis. It also contributes to other biological processes including vascular physiology, pain, itch, and cancer (Gupta et al. Mast Cells-Neural Interactions Contribute to Pain and Itch 282 (2018) 168-187).

Itch and pain are closely related with similarities and differences. At the fundamental level, itch is exclusively sensed as an irritation in the skin, while pain can be experienced in almost all the body parts. Itch can be classified into four categories, 1) pruriceptive itch, itch that originates from the skin and perhaps the most common symptom in dermatology, as a result of dry skin, atopic eczema, psoriasis, urticaria, scabies or other inflammatory skin diseases, 2) neuropathic itch, caused by nerve damage, 3) neurogenic itch, caused by dysfunction of the nervous system in the absence of peripheral neuropathy, and 4) psychological or psychiatric itch, due to psychological abnormalities. Similarly, pain can also be classified into four categories, 1) nociceptive pain, caused by activation of peripheral nerve fibers by noxious stimulation, 2) inflammatory pain, pain associated with tissue damage and inflammation, 3) neuropathic pain, caused by damage or disorder affecting of the nervous system, 4) Psychogenic pain, caused by mental, emotional, or behavioral factors.

The sensory neurons get stimulated when an injury occurs and generate an action potential. Pain signals are transmitted to the brain through the sensory nerves, through several receptors (Afridi et al. Pain Perception and Management: Where do We Stand? Curr Mol Pharmacol. 2021; 14 (5) 678-688). This process is also known as nociception. The Transient Receptor Potential (TRP-(TRPV1, TRPA1), Toll-like Receptors (TLRs), and protease activated receptors (PARs) family, constitute the major groups of molecular transducers of pain (Mickle et al. Nociceptive TRP Channels: Sensory Detectors and Transducers in Multiple Pain Pathologies. Pharmaceuticals. 2016; 9 (4): 72; Liu et al. New insights into the mechanisms of itch: are pain and itch controlled by distinct mechanisms? Pflugers Arch 465 (2013) 1-24).

Currently, there are very limited treatment options available for managing pain and allergy such as, opioid analgesics, Non-steroidal Anti-inflammatory Drugs (NSAID), antidepressants, and cannabinoids depending on the severity of the condition. The inadequate treatment options available for chronic pain also contribute to overuse of these drugs, causing several long-term adverse effects. Antihistamines are the preferred choice of treatment for the management of allergy. There exists an unmet need to find a natural plant-based alternative that acts dually by managing symptoms associated with pain and allergy.

Plant extracts standardized to contain different active ingredients have been reported for managing pain and allergy. EP 3409275 discloses a composition comprising Palmitoylethanolamide and curcumin for managing inflammation and/or hyperalgesia. Guineesine has also been reported to exert anti-inflammatory effects (Moreno et al., An endocannabinoid uptake inhibitor from black pepper exerts pronounced anti-inflammatory effects in mice, J Agric Food Chem. 2017 Nov. 1; 65 (43): 9435-9442). However, a need to find an effective and synergistic composition for managing both pain and allergy is still warranted and remains an unsolved problem. The present invention solves the above-mentioned problem by disclosing a composition comprising Guineesine and β-Caryophyllene (BCP), along with Palmitoylethanolamide (PEA) for the management of pain and allergy.

It is a general objective of the invention to disclose a composition comprising 4-10% w/w Guineesine (BPE) and β-Caryophyllene (BCP), wherein the composition further comprises Palmitoylethanolamide (PEA).

It is another objective of the invention, to disclose a method of management of pain in a subject, comprising steps of identifying the subject in need of pain management and administering an effective dose of a composition comprising 4-10% w/w Guineesine (BPE) and β-Caryophyllene (BCP) to the subject to alleviate pain. The composition further comprises Palmitoylethanolamide (PEA).

It is another objective of the invention, to disclose a method for managing allergy in a subject, comprising identifying the subject in need of allergy management and administering an effective dose of a composition comprising 4-10% w/w Guineesine (BPE) and β-Caryophyllene (BCP) to the subject to alleviate allergy. The composition further comprises Palmitoylethanolamide (PEA).

In yet another objective, the invention covers use of a composition comprising 4-10% w/w Guineesine (BPE) and β-Caryophyllene (BCP) for management of pain in a subject, wherein the composition further comprises Palmitoylethanolamide.

In yet another objective, the invention covers use of a composition comprising 4-10% w/w Guineesine (BPE) and β-Caryophyllene (BCP) for management of allergy in a subject, wherein the composition further comprises Palmitoylethanolamide (PEA).

In yet another objective, the invention covers a composition comprising 4-10% w/w Guineesine (BPE) and β-Caryophyllene (BCP) for use in alleviating pain in a subject, wherein the composition further comprises Palmitoylethanolamide (PEA).

In yet another objective, the invention covers a composition comprising 4-10% w/w Guineesine (BPE) and β-Caryophyllene (BCP), for use in alleviating allergy in a subject, wherein the composition further comprises Palmitoylethanolamide (PEA).

### SUMMARY OF THE INVENTION

The invention broadly solves the problems mentioned in the background by covering a composition comprising 4-10% w/w Guineesine (BPE), β-Caryophyllene (BCP), and Palmitoylethanolamide (PEA) The invention further covers a method, use of composition for treating pain and allergy in a subject using the said composition.

The first aspect of the invention relates to a composition comprising 4-10% w/w Guineesine and β-Caryophyllene.

The second aspect of the invention covers a method of managing pain in a subject, said method comprising steps of: identifying the subject in need of pain management administering to the subject an effective dose of a composition comprising 4-10% w/w Guineesine and β-Caryophyllene to alleviate pain. In a related aspect, the composition further comprises Palmitoylethanolamide.

Yet another aspect of the invention covers use of a composition comprising 4-10% w/w Guineesine (BPE) and β-Caryophyllene (BCP) for management of pain in a subject. In a related aspect, the composition further comprises Palmitoylethanolamide.

In yet another aspect of the invention covers a composition comprising 4-10% w/w Guineesine (BPE) and β-Caryophyllene (BCP), further comprising Palmitoylethanolamide (PEA) for use in alleviating pain in a subject.

In yet another aspect of the invention covers a method of managing allergy in a subject, said method comprising steps of identifying the subject in need of allergy management; administering to the subject an effective dose of a composition comprising 4-10% w/w Guineesine (BPE) and β-Caryophyllene (BCP) to alleviate allergy. In a related aspect, the composition further comprises Palmitoylethanolamide.

In yet another aspect of the invention, use of a composition comprising 4-10% w/w Guineesine (BPE) and β-Caryophyllene (BCP) for managing allergy in a subject. In a related aspect, the composition further comprises Palmitoylethanolamide.

In yet another aspect of the invention, covers a composition comprising 4-10% w/w Guineesine (BPE) and β-Caryophyllene (BCP), further comprising Palmitoylethanolamide (PEA) for use in alleviating allergy in a subject.

The broader scope of applicability of the present invention will be apparent from the detailed description below. However, it should be understood that the detailed description and specific examples, while indicating preferred embodiments of the invention, should not be construed as the limitations to the invention, and it is within the scope of those skilled in the art to make various changes and modifications, such as changing the concentration range of composition used, derivatives/analogs of composition used herewith, experimental conditions, choice of mammals, to the spirit and scope of the invention from this detailed description.

### BRIEF DESCRIPTION OF DRAWINGS

FIGS. 1A, 1B, 1C and 1D are graphical representations of dose response suggesting inhibition of intracellular calcium levels using 4-10% w/w Guineesine (BPE) (FIG. 1A), β-Caryophyllene (BCP) (FIG. 1B), and Palmitoylethanolamide (PEA) (FIG. 1C), combination of BPE, BCP, and PEA (FIG. 1D)

FIG. 2A, 2B, 2C are graphical representations of dose response suggesting inhibition of inducible nitric oxide using 4-10% w/w Guineesine (BPE) (FIG. 2A), β-Caryophyllene (BCP) (FIG. 2B), combination of BPE, BCP, and PEA (FIG. 2C).

FIGS. 3A, 3B, 3C and 3D are graphical representations of a dose response suggesting inhibition of Interleukin-6 (IL-6) using 4-10% w/w Guineesine (BPE) (FIG. 3A), β-Caryophyllene (BCP) (FIG. 3B), and Palmitoylethanolamide (PEA) (FIG. 3C), combination of BPE, BCP, and PEA (FIG. 3D).

FIGS. 4A, 4B, 4C and 4D are graphical representations of a dose response suggesting inhibition of histamine release using 4-10% w/w Guineesine (BPE) (FIG. 4A), β-Caryophyllene (BCP) (FIG. 4B), and Palmitoylethanolamide (PEA) (FIG. 4C), combination of BPE, BCP, and PEA (FIG. 4D).

FIG. 5 shows inhibition of TNF-α using the samples (Values are expressed as Mean±S.E. Number of animals in each group: 6, *P<0.05, **P<0.001

FIG. 6 shows inhibition of IL-1β using the samples (Values are expressed as Mean±S.E. Number of animals in each group: 6, **P<0.001).

FIG. 7 shows the western blot image for the quantifying the expression of TRPV1 and COX-2 in the spinal cord of animals administered with 4-10% w/w Guineesine (BPE), β-Caryophyllene (BCP), and Palmitoylethanolamide (PEA) and combination of BPE, BCP, and PEA. GAPDH is used as a house keeping gene.

FIG. 8 shows Tail-flick test in animal model to assess pain (Values are expressed as Mean±S.E. Number of animals in each group: 6, p<0.001).

FIG. 9 shows Hot plate test in animal model to assess pain (Values are expressed as Mean±S.E. Number of animals in each group: 6, p<0.001).

FIG. 10 shows effect of samples in organ weight (Values are expressed as Mean±S.E. Number of animals in each group: 6, #Compared to control P<0.001, * compared to Ova, *P<0.01, **P<0.001, ***P,0.0001).

FIG. 11 shows effect of samples on nasal symptoms, nose rubbing (Values are expressed as Mean±S.E. Number of animals in each group: 6, #Compared to control P<0.001, * compared to Ova, *P<0.01, **P<0.001, ***P,0.0001).

FIG. 12 shows effect of samples on nasal symptoms, sneezing (Values are expressed as Mean±S.E. Number of animals in each group: 6, #Compared to control P<0.001, * compared to Ova, *P<0.01, **P<0.001, ***P,0.0001).

FIG. 13 is nasal redness score (Values are expressed as Mean±S.E. Number of animals in each group: 6, *P<0.01, **P<0.001, ***P<0.0001).

FIG. 14 shows total cell count of Bronchoalveolar lavage Fluid (BALF) (Values are expressed as Mean±S.E. Number of animals in each group: 6, #Compared to control P<0.01, ##p<0.001, * compared to Ova, *P<0.01, **P<0.001, ***P,0.0001).

FIG. 15 shows differential count of Macrophages, neutrophil, eosinophil, and lymphocyte in Bronchoalveolar lavage Fluid (BALF) (Values are expressed as Mean±S.E. Number of animals in each group: 6, #Compared to control P<0.01, ##p<0.001, * compared to Ova, *P<0.01, **P<0.001, ***P,0.0001).

FIG. 16 shows Histamine content in Bronchoalveolar lavage Fluid (BALF) (Values are expressed as Mean±S.E. Number of animals in each group: 6, #Compared to control P<0.01, ##p<0.001, * compared to Ova, *P<0.01, **P<0.001, *** P,0.0001).

### DESCRIPTION OF PREFERRED EMBODIMENTS

### Selected Definitions

All the terms used in this application carry ordinary meaning as known in the prior art unless otherwise specified. Few other specific definitions used in this invention are explained below, which applies throughout this specification. Claims provide broader definition unless and otherwise specified.

Furthermore, the terms “approximately,” “approximate,” “about,” and similar terms generally refer to ranges that include the identified value within a margin of 20%, 10%, or preferably 5%, and any values there between.

As used herein, treatment or management of a condition refers to effectively ameliorating conditions disclosed in the invention. An effective dose refers to positive or modulatory effects of a condition in a subject covered under this invention.

As used herein, condition refers to pain or allergy and associated symptoms disclosed in the invention.

As used herein, Guineesine is also referred to as Guineensine, with CAS no. is 55038-30-7.

As used herein, % w/w refers to purity of the component.

As used herein, BPE is 4-10% w/w Guineesine.

As used herein, BCP is β-Caryophyllene.

As used herein, PEA is Palmitoylethanolamide.

The invention covers a composition comprising 4-10% w/w Guineesine and β-Caryophyllene. The invention further covers a method, use of composition for treating pain and allergy.

In the most preferred embodiment, the invention discloses a composition comprising 4-10% w/w Guineesine and β-Caryophyllene. In a related embodiment of the invention, the composition further comprises Palmitoylethanolamide. In a related embodiment of the invention, the composition can be used individually or in any possible combination. In related embodiment of the invention, the composition is formulated with pharmaceutically/nutraceutically acceptable excipients, adjuvants, diluents, or carriers, and administered orally in the form of tablets, capsules, syrups, gummies, powders, suspensions, emulsions, chewables, candies, and eatables.

In another most preferred embodiment of the invention, the invention discloses a method for of managing pain in a subject, comprising steps of identifying the subject in need of pain management; and administering to the subject, an effective dose of a composition comprising 4-10% w/w Guineesine and β-Caryophyllene, wherein the treatment alleviates pain in the subject.

In a related embodiment of the invention, the composition further comprises Palmitoylethanolamide. In a related embodiment of the invention, the composition can be used individually or in combination. In a related embodiment of the invention, the effective dose of the composition in vitro is in the range 0.1-20 μg/mL and the effective dose in vivo is in the range 1-30 mg/Kg. In a related embodiment of the invention, wherein the composition alleviates the pain selected from the group consisting of nociception, inflammation, and neuropathic. In another related embodiment, the manifestation of pain results in conditions selected from hyperalgesia, allodynia. In a related embodiment of the invention, wherein the alleviation of pain is mediated by, decrease of COX-2 expression, inhibition of intracellular calcium release by modulating the expression of TRPV receptors, inhibition of inducible Nitric oxide, inhibition of IL-6, and inhibition of histamine release from mast cell degranulation. In a related embodiment of the invention, wherein the subject is selected from the group consisting of a mammalian cell or mammal. In a related embodiment of the invention, wherein the composition is formulated with pharmaceutically/nutraceutically acceptable excipients, adjuvants, diluents, or carriers, and administered orally in the form of tablets, capsules, syrups, gummies, powders, suspensions, emulsions, chewables, candies, and eatables.

In another most preferred embodiment of the invention, the invention discloses a method for of managing allergy in a subject, comprising steps of: identifying the subject in need of allergy management; and administering to the subject, an effective dose of a composition comprising 4-10% w/w Guineesine and β-Caryophyllene; wherein the treatment alleviates allergy in the subject.

In a related embodiment of the invention, the composition further comprises Palmitoylethanolamide. In a related embodiment of the invention, wherein the composition can be used individually or in combination. In a related embodiment of the invention, wherein effective dose in vitro is in the range 0.1-20 μg/mL and the effective dose in vivo is in the range 1-30 mg/Kg. In a related embodiment of the invention, wherein the composition alleviates allergy mediated by allergens selected from the group consisting of eggs, milk, mustard, peanuts, crustaceans and mollusks, fish, sesame seeds, soy, sulphites, tree nuts, wheat and triticale, histamine rich foods, ovalbumin, environmental pollutant and chemicals, antibiotics, and drugs. In a related embodiment of the invention, wherein the alleviation of allergy is mediated by inhibition of histamine release from mast cells, reduction in nasal symptoms and reduction of bronchoalveolar lavage fluid (BALF) cell count. In a related embodiment of the invention, wherein the subject is selected from the group consisting of a mammalian cell or a mammal. In a related embodiment of the invention, wherein the composition is formulated with pharmaceutically/nutraceutically acceptable excipients, adjuvants, diluents, or carriers, and administered orally in the form of tablets, capsules, syrups, gummies, powders, suspensions, emulsions, chewables, candies, and eatables.

In yet another most preferred embodiment of the invention, the invention discloses use of a composition in managing pain in a subject comprising steps of identifying the subject in need of pain management; and administering to the subject, an effective dose of a composition comprising 4-10% w/w Guineesine and β-Caryophyllene, wherein the treatment alleviates pain in the subject.

In a related embodiment of the invention, the composition further comprises Palmitoylethanolamide. In a related embodiment of the invention, wherein the composition can be used individually or in combination. In a related embodiment of the invention, wherein effective dose in vitro is in the range 0.1-20 μg/mL and the effective dose in vivo is in the range 1-30 mg/Kg. In a related embodiment of the invention, wherein the composition alleviates the pain selected from the group consisting of nociception, inflammation, and neuropathic. In another related embodiment, the manifestation of pain results in conditions selected from hyperalgesia, allodynia. In a related embodiment of the invention, wherein the alleviation of pain is mediated by, decrease of COX-2 expression, inhibition of intracellular calcium release by modulating the expression of TRPV receptors, inhibition of inducible nitric oxide, inhibition of IL-6, and inhibition of histamine release from mast cell degranulation. In a related embodiment of the invention, wherein the subject is selected from the group consisting of a mammalian cell or mammal. In a related embodiment of the invention, wherein the composition is formulated with pharmaceutically/nutraceutically acceptable excipients, adjuvants, diluents, or carriers, and administered orally in the form of tablets, capsules, syrups, gummies, powders, suspensions, emulsions, chewables, candies, and eatables.

In yet another most preferred embodiment of the invention, the invention discloses use of a composition in managing allergy in a subject, comprising steps of: identifying the subject in need of pain management; and administering to the subject, an effective dose of a composition comprising 4-10% w/w Guineesine and β-Caryophyllene, wherein the treatment alleviates pain in the subject.

In a related embodiment of the invention, the composition further comprises Palmitoylethanolamide. In a related embodiment of the invention, wherein the composition can be used individually or in any possible combination. In a related embodiment of the invention, wherein effective dose in vitro is in the range 0.1-20 μg/mL and the effective dose in vivo is in the range 1-30 mg/Kg. In a related embodiment of the invention, wherein the composition alleviates allergy mediated by allergen selected from the group consisting of eggs, milk, mustard, peanuts, crustaceans and molluscs, fish, sesame seeds, soy, sulphites, tree nuts, wheat and triticale, histamine rich foods, ovalbumin, environmental pollutant and chemicals, antibiotics, and drugs. In a related embodiment of the invention, wherein the alleviation of allergy is mediated by inhibition of histamine release from mast cells reduction in nasal symptoms and reduction of bronchoalveolar lavage fluid (BALF) cell count. In a related embodiment of the invention, wherein the subject is selected from the group consisting of a mammalian cell or mammal. In a related embodiment of the invention, wherein the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

In yet another most preferred embodiment of the invention, the invention discloses a composition comprising 4-10% w/w Guineesine and β-Caryophyllene for use in alleviating pain in a subject. In a related embodiment of the invention, the composition further comprises Palmitoylethanolamide. In a related embodiment of the invention, wherein the composition can be used individually or in combination. In a related embodiment of the invention, wherein effective dose in vitro is in the range 0.1-20 μg/mL and the effective dose in vivo is in the range 1-30 mg/Kg. In a related embodiment of the invention, wherein the composition alleviates the pain selected from the group consisting of nociception, inflammation, and neuropathic. In another related embodiment, the manifestation of pain results in conditions selected from hyperalgesia, allodynia. In a related embodiment of the invention, wherein the alleviation of pain is mediated by decrease of COX-2 expression, inhibition of intracellular calcium release by modulating the expression of TRPV receptors, inhibition of inducible Nitric oxide, inhibition of IL-6, and inhibition of histamine release from mast cell degranulation. In a related embodiment of the invention, wherein the subject is selected from the group consisting of a mammalian cell or mammal. In a related embodiment of the invention, wherein the composition is formulated with pharmaceutically/nutraceutically acceptable excipients, adjuvants, diluents, or carriers, and administered orally in the form of tablets, capsules, syrups, gummies, powders, suspensions, emulsions, chewables, candies, and eatables.

In yet another most preferred embodiment of the invention, the invention discloses a composition comprising: 4-10% w/w Guineesine and β-Caryophyllene for use in alleviating allergy in a subject. In a related embodiment of the invention, the composition further comprises Palmitoylethanolamide. In a related embodiment of the invention, wherein the composition can be used individually or in combination. In a related embodiment of the invention, wherein effective dose in vitro is in the range 0.1-20 μg/mL and the effective dose in vivo is in the range 1-30 mg/Kg. In a related embodiment of the invention, wherein the composition alleviates allergy mediated by allergens selected from the group consisting of eggs, milk, mustard, peanuts, crustaceans and mollusks, fish, sesame seeds, soy, sulphites, tree nuts, wheat and triticale, histamine rich foods, ovalbumin, environmental pollutant and chemicals, antibiotics and drugs. In a related embodiment of the invention, wherein the alleviation of allergy is mediated by inhibition of histamine release from mast cells reduction in nasal symptoms and reduction of bronchoalveolar lavage fluid (BALF) cell count. In a related embodiment of the invention, wherein the subject is selected from the group consisting of a mammalian cell and mammal. In a related embodiment of the invention, wherein the composition is formulated with pharmaceutically/nutraceutically acceptable excipients, adjuvants, diluents, or carriers, and administered orally in the form of tablets, capsules, syrups, gummies, powders, suspensions, emulsions, chewables, candies, and eatables.

### EXAMPLES

The examples are provided solely for illustration and are not to be construed as limitations of the present disclosure, as many variations thereof are possible without departing from the spirit and scope of the present disclosure.

### Example 1: Materials in the Composition

As used herein, samples refer to 4-10% w/w Guineesine (BPE), β-Caryophyllene (BCP), or Palmitoylethanolamide (PEA) or combination thereof as disclosed in respective embodiments.

As used herein, BPE is 4-10% w/w Guineesine. Guineesine is found in different pepper species such as Piper nigrum, Piper longum, Piper guineense, Piper officinarum and Piper sylvaticum. As an illustrative example, the below process discloses a method to isolate 4-10% w/w Guineesine from black pepper (Piper nigrum).
a. Taken 12.0 Kg of Piperine Reduced Pepper Oleoresin obtained after piperine extraction process in a clean reactor,
b. Charged 70 L hexane in the same reactor and stirred for 1 hr. at 50-55° C. and allowed to cool at room temperature.
c. Removed the upper hexane layer and concentrated to recover the hexane.
d. Charged 70 L of Ethyl acetate to the hexane removed residue to make it clear solution.
e. Charged 70 L of DM water and give mild agitation for 5 minutes to remove the water layer.
f. Repeated the above steps once.
g. Concentrated the Ethyl acetate layer fully under vacuum and added 12 L mixture of Hexane:Ethyl acetate (6:4) and collected the concentrated mass in a carboy.
h. Packed a Column with 100 Kg of Silica gel (60-120 mesh) by using 200 L of hexane:ethyl acetate (6:4) as mobile phase.
i. Loaded the concentrated mass obtained from step g.
j. Started elution with isocratic mobile phase (hexane 6:ethyl acetate 4) and collected four fractions (100 L each) and concentrated all four fractions separately.
Fraction 1: Concentrated, recovered the solvent and discarded the residue.
Fraction 2: After concentration yield: 500 g Paste and Assay of Pellitorine: 20-25%
Fraction 3: After concentration yield: 1.5 Kg Paste and Assay of Guineesine: 4-10%
Fraction 4: After concentration discarded the residue (not containing Piperine, Pellitorine and Guineesine).
Washed the column with ethyl acetate, concentrate, Yield: 5.0 Kg, Assay of Piperine 25-30% by HPLC. Fraction 3 (Guineesine: 4-10%) was concentrated and used for further experiments, which is devoid of piperine.

As used herein, BCP is β-Caryophyllene. BCP can be isolated from Piper nigrum, Syzygium aromaticum, Origanum vulgare, Copaifera langsdorffii, Chromolaena odorata, Humulus lupulus, and Cinnamomum spp. As an illustrative example, BCP was isolated from black pepper using the below process: Charged 1000 Kg of Black pepper coarse powder in a cleaned extractor equipped with hydro-distillation setup.
a. Started distillation by heating and purging steam, collected the water & oil mixture and separated the clear oil.
b. Passed the oil through anhydrous sodium sulphate to remove moisture. Oil yield: 15 Kg (1.5%), Beta-Caryophyllene content: 15-20% w/w)
c. Charged above 15 Kg of Black pepper oil in a cleaned reactor equipped with high vacuum distillation setup.
d. Then started high vacuum distillation for collecting low volatile fraction (Temperature 100° C.-110° C., Vacuum 120 mm Hg) for 8-10 hrs.
e. Yield of low volatile fraction: 10-11 kg.
f. Continue the high vacuum distillation of residual fraction by increasing the temperature. (Temperature 150° C.-160° C., Vacuum 1 mm Hg)
g. Collect and combine the Beta-Caryophyllene enriched fraction monitoring by GC. Yield: 3-4 kg (Expected Assay: 70-80%)
h. The Enriched fraction was again subjected to high vacuum distillation at 160° C. under 1 mm Hg vacuum.
i. Collected the distillate to characterize Beta-Caryophyllene >90% fraction: 2.0 Kg.

As used herein, PEA is Palmitoylethanolamide. In all embodiments of the invention, Palmitoylethanolamide (PEA) is chemically synthesized.

### Example 2: Use of Composition Comprising and 4-10% w/w Guineesine (Specifically 6% Guineesine), β-Caryophyllene (BCP), and Palmitoylethanolamide (PEA) for the Management of Pain-In-Vitro Experiments

Mouse macrophage cell line (RAW264.7) was purchased from National Center for Cell Science (NCCS, Pune, India) and maintained as a monolayer culture in Dulbecco's modified minimal essential medium (DMEM, Life Technologies, CA, USA) supplemented with 10% (v/v) fetal bovine serum (FBS; GIBCO/Invitrogen, Carlsbad, CA) at 37° C. in a humidified 5% CO₂ incubator. The composition was used in the ratio of 1:1:0.3, however it is evident to the person skilled in the art that it is obvious to use any possible ratios to arrive at the desired results. The composition comprising 4-10% w/w Guineesine, β-Caryophyllene, and Palmitoylethanolamide in the ratios of 1-2:0.5-1:0.3-1 parts respectively, or preferably in the ratio of 1:0.5:0.5 parts respectively, or preferably in the ratio of 1:0.5:0.15 parts respectively, or preferably in the ratio of 1:1:0.33 parts respectively are also covered.

### Inhibition of Intracellular Calcium

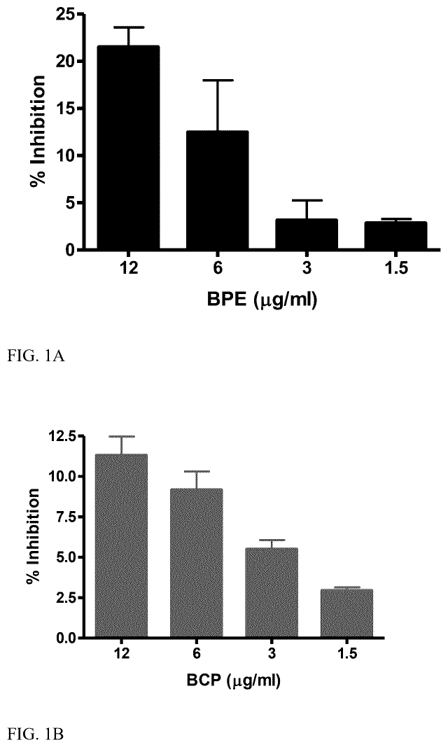

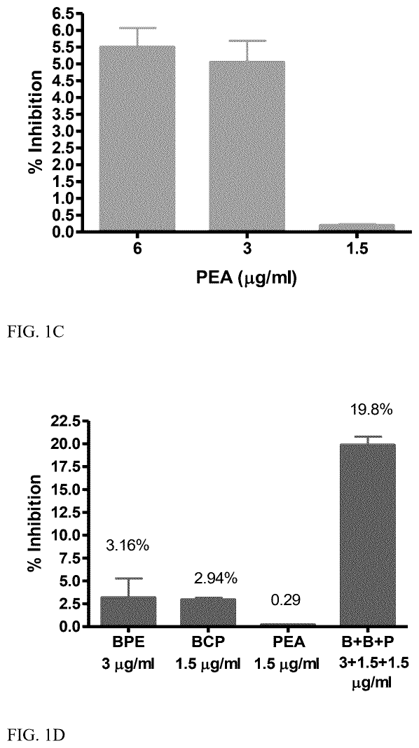

Various environmental stimuli or neurotransmitters often cause changes in intracellular calcium concentration in the skin. Keratinocytes, primary cell type found in the outermost layer of the skin. Epidermal keratinocytes express functional sensory receptors, such as TRPV1, TRPV3, and TRPV4. Pain stimulus increases intracellular calcium through TRPV channels. Hydrogen peroxide mediated calcium stimulation was tested. Human epidermal keratinocyte cells (HaCaT) (5×10⁴) were seeded in 96 black well plate for overnight. Samples were Pre-treated for 2 hours in 1% DMEM. Cells were stimulated with 10 mM H₂O₂ for 30 mins, and Fluo-8AM dye was added (HBBS buffer) for 1 hr. After incubation, cells were washed with PBS twice and the fluorescence was measured at the Ex/Em of 485/520 nm. Clearly, 6% w/w Guineesine, β-Caryophyllene, Palmitoylethanolamide wherein each showed inhibition of intracellular calcium levels (FIGS. 1A, 1B, and 1C) and even more so the combination of these three showed synergistic activity (FIG. 1D).

### Evaluation of Immune Markers

After tissue damage, mast cells and macrophages are activated and some blood-borne immune cells, including neutrophils, may be recruited. Various immune mediators are released (such as tumor necrosis factor-α (TNFα), interleukin-1β (IL-1β), interleukin-6 (IL-6), inducible nitric oxide (NO), bradykinin, nerve growth factor (NGF) and protons), which exert their effects by acting directly on nociceptors or indirectly through the release of other mediators, most notably prostanoids (Marchand et al. Role of the Immune System in Chronic Pain, Nature Reviews 6 (2005) 521-532).

### Inhibition of Inducible Nitric Oxide

Nitric oxide (NO) is involved in the mechanisms of pain generation and transmission in the central, peripheral nervous systems, and locally released pain mediators (inflammation and vascular edema). They are one of the pro-inflammatory cytokines released when immune cells are activated. The Nitrite estimation was carried out using the Griess reaction. To the 50 μL of cell supernatant, Griess reagent I (50 μL) and Griess reagent II (50 μL) were added and incubated for 10 min at room temperature and absorbance were read at 540 nm using Microplate reader.

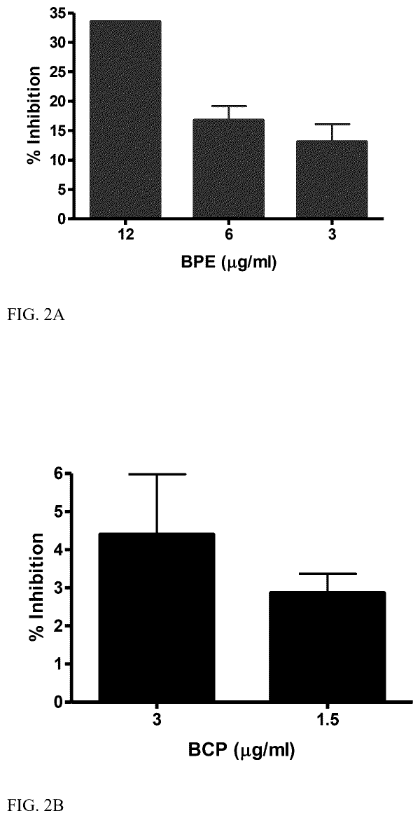

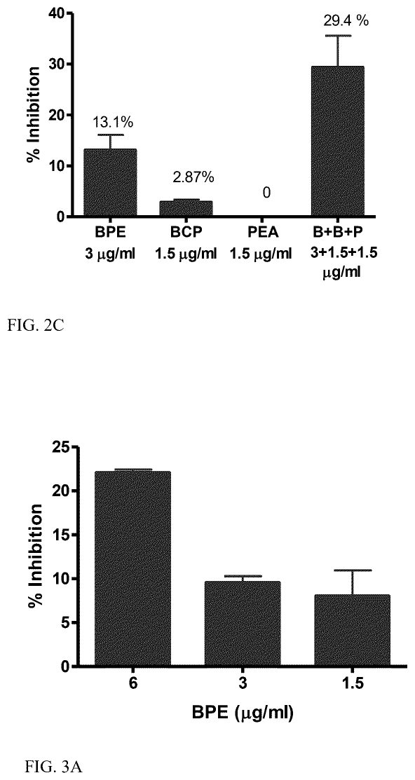

It is evident that 6% w/w Guineesine, β-Caryophyllene, each showed inhibition of nitric oxide levels (FIG. 2A, 2B). The combination of Black pepper extract (6% Guineesine), β-Caryophyllene and Palmitoylethanolamide showed a synergistic inhibition of nitric oxide in macrophages (FIG. 2C).

### Inhibition of IL-6

IL-6 is implicated in neuropathic pain. Moreover, in a model of neuropathic pain, IL-6-knockout mice showed less thermal hyperalgesia and mechanical allodynia compared with wild-type mice. In CNS, microglia activation is likely the first step in a cascade of immune response and could be responsible for neuropathic pain states. P2X7 are expressed on microglia and their activation by ATP lead to the production and release of inflammatory cytokines. Mice that lacked the receptor showed impaired ability to develop neuropathic pain. (Zhuang et al/ERK is sequentially activated in neurons, microglia, and astrocytes by spinal nerve ligation and contributes to mechanical allodynia in this neuropathic pain model. Pain 114, (2005) 149-159; Chessell et al. Disruption of the P2X7 purinoceptor gene abolishes chronic inflammatory and neuropathic pain. Pain 114, 386-396 (2005); Marchand et al. Role of the Immune System in Chronic Pain, Nature Reviews 6 (2005) 521-532).

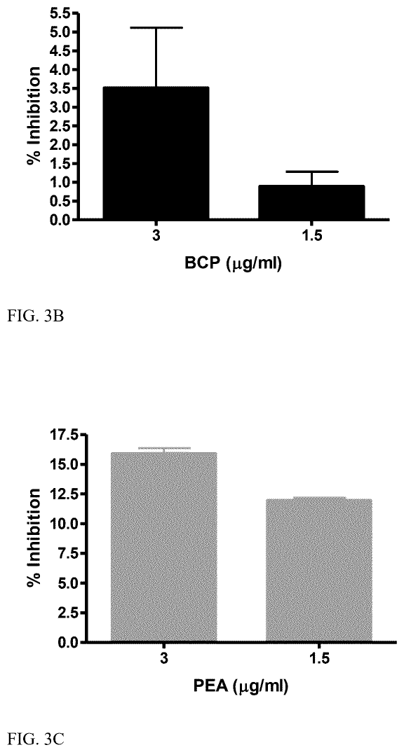

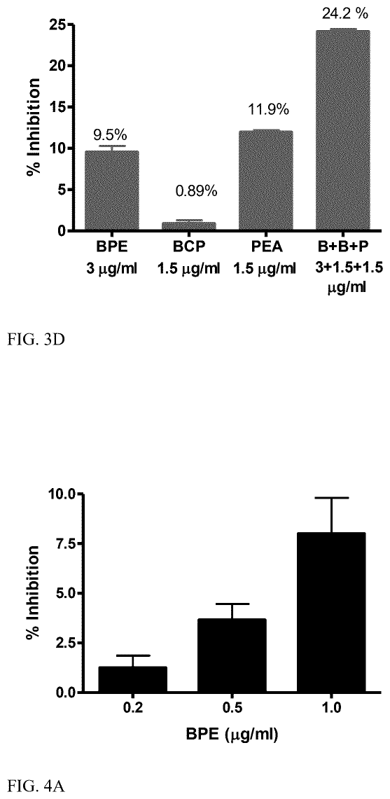

RAW 264.7 (5×10⁵ cells/well) macrophage cells were induced with LPS (100 ng/mL) along with or without different concentrations of Samples. The supernatants were used for IL6 estimation using ELISA kits as per the manufacturer's instruction (R&D Systems, Minneapolis, Minnesota, USA). Black pepper extract (6% Guineesine) and Palmitoylethanolamide (PEA) showed dose dependent inhibition of IL6 induced by LPS (FIG. 3A, 3B), β-Caryophyllene showed lower activity (FIG. 3B). The combination of Black pepper extract (6% w/w Guineesine), PEA, and BCP showed a synergistic inhibition of IL-6 in macrophages (FIG. 3D).

### Inhibition of Histamine Release

Histamine (2-(4-imidazolyl)-ethylamine)), is one of the neurotransmitters involved in various physiological and pathological processes. Histamine is released by neuronal and non-neuronal sources and is responsible for the contraction of smooth muscles of the lungs, uterus, and intestine, secretion of gastric acid in the stomach, vasodilation, and modulation of heart rate and contractility. Histamine is one of the products of degranulation of the inflammation induced mast cell (Yam et al. General Pathways of Pain Sensation and the Major Neurotransmitters Involved in Pain Regulation, Int. J. Mol. Sci. 19 (2018) 1-23). It acts via several receptors (H1R, H2R, H3R, and H4R) and plays a critical role in neurogenic inflammation and pain transmission in the periphery and CNS. It has been shown that antagonists of H1-H4 receptors inhibit neurogenic inflammation, pain, mechanical and/or thermal hyperalgesia, and itch in animal models in a case-specific manner (Gupta et al. Mast Cell-neural interactions Contribute to Pain and Itch, Immunological Reviews 282 (2018) 168-187; Rosa et al. The Role of Histamine in Neurogenic Inflammation. Br. J. Pharmacol. 170 (2013) 38-45).

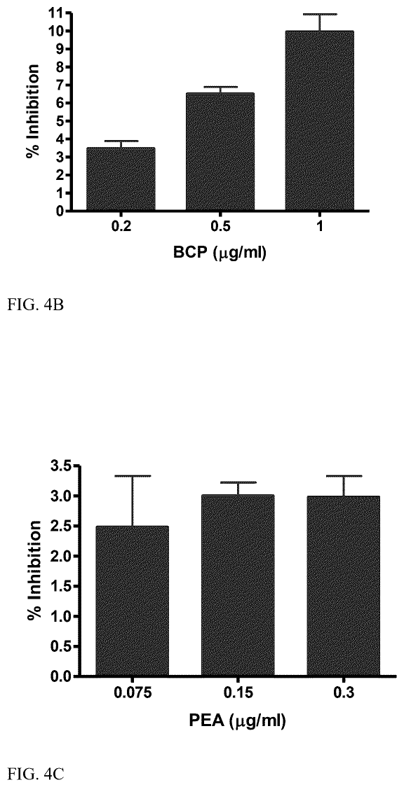

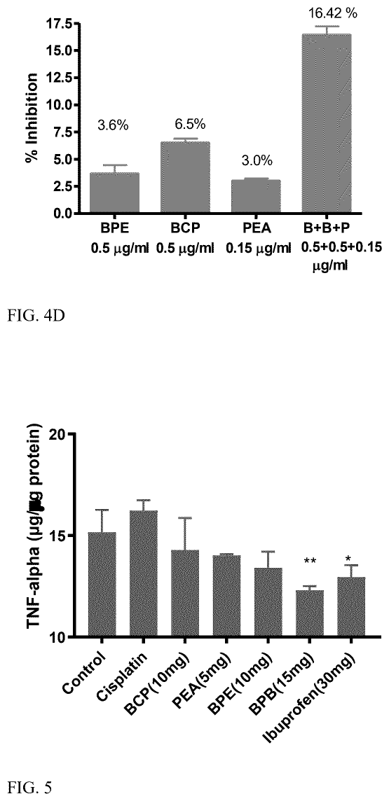

Mouse mast cells P815 (5×105) were plated in 48 well plates and washed with tyrode's buffer. After washing, cells were treated for 30 min with 80 μg/ml compound C48/80, which is a mixed polymer of p-methoxy-N-methyl phenylethylamine crosslinked by formaldehyde and widely used in animal and tissue models as a “selective” mast cell activator, containing indicated concentrations of Samples (FIGS. 4A-4D). Reaction was terminated by placing the cells on ice for 10 mins and centrifuged for 5 minutes at 2400 rpm. 100 μL of cell supernatant was mixed with 50 μL of 4N NaOH and 10 μL of O-Phthaldialdehyde (OPT, 1 mg·mL-1 in methanol, Sigma). By incubation at room temperature for 10 min, 50 μL of 1N HCl was added to stop the reaction. The fluorescence was measured at Ex/Em of 390/460 nm using a microplate reader (FLOUSTAR).

Black pepper extract (6% Guineesine) and β-Caryophyllene showed dose dependent inhibition of histamine release from mast cells (FIG. 4A, 4B). Palmitoylethanolamide showed lower activity (FIG. 4C). The combination of Black pepper extract (6% Guineesine) and β-Caryophyllene and Palmitoylethanolamide showed a synergistic inhibition (FIG. 4D) of histamine release from mast cells.

### Example 3: Use of Composition Comprising 6-10% Guineesine (Specifically 6% Guineesine), β-Caryophyllene, and Palmitoylethanolamide for the Management of Pain-In-Vivo Experiments

As an illustrative example to evaluate the effect of the composition on reducing pain, in-vivo experiments were carried out in Cisplatin induced peripheral neuropathy animal models. Peripheral neuropathy, a frequent side effect of cancer chemotherapeutics, includes spontaneous pain and mechanical/cold allodynia. This is referred to as Chemotherapy induced peripheral neuropathy (CIPN). CIPN caused by cisplatin is reported to involve mitochondria damage, oxidative stress, inflammation, and activation of transient receptor potential vanilloid channels (TRPV). For the animal studies, C57BL/6 mice (either sex) weighing 22-25 g were used with 6 animals selected in each group as listed and treatment regime is listed across groups in Table 1, The composition was used in the ratio of 1:1:0.3, however it is evident to the person skilled in the art that it is obvious to use any possible ratios to arrive at the expected results. The composition comprising Guineesine, β-Caryophyllene, and Palmitoylethanolamide in the ratios of 1-2:0.5-1:0.3-1 parts respectively, or preferably in the ratio of 1:0.5:0.5 parts respectively, or preferably in the ratio of 1:0.5:0.15 parts respectively, or preferably in the ratio of 1:1:0.33 parts respectively are also covered.

TABLE 1
Animal groups for Pain Study

| | |
|---|---|
| Group 1 | Normal Control |
| Group 2 | Disease Control: Platinum (Cisplatin 3 mg/kg bodyweight) was |
| | administered intraperitoneally on the experiment's 0^{th}, 2^{nd}, |
| | 4^{th}, and 6^{th} days. |
| Group 3 | Platinum (Cisplatin 3 mg/kg bodyweight) administered |
| | intraperitoneally on the 0^{th}, 2^{nd}, 4^{th}, and 6^{th} days. The Test Drug |
| | (β-Caryophyllene 10 mg/kg) given orally for 21 days |
| Group 4 | Platinum (Cisplatin 3 mg/kg bodyweight) administered |
| | intraperitoneally on the 0^{th}, 2^{nd}, 4^{th}, and 6^{th} days. The Test Drug |
| | (Palmitoylethanolamide 10 mg/kg) given orally for 21 days |
| Group 5 | Platinum (Cisplatin 3 mg/kg bodyweight) administered |
| | intraperitoneally on the 0^{th}, 2^{nd}, 4^{th}, and 6^{th} days. The Test Drug |
| | BPE, (6% Guineesine- 10 mg/kg) given orally for 21 days |
| Group 6 | Platinum (Cisplatin 3 mg/kg bodyweight) administered |
| | intraperitoneally on the 0^{th}, 2^{nd}, 4^{th}, and 6^{th} days. The Test Drug |
| | (Combination (BEP + BCP + PEA) 15 mg/kg) given orally |
| | for 21 days |
| Group 7 | Platinum (Cisplatin 3 mg/kg bodyweight) administered |
| | intraperitoneally on the 0^{th}, 2^{nd}, 4^{th}, and 6^{th} days. Ibuprofen |
| | (30 mg/Kg) given orally for 21 days. |

### Assessment of Inflammatory Cytokines and Biomarkers

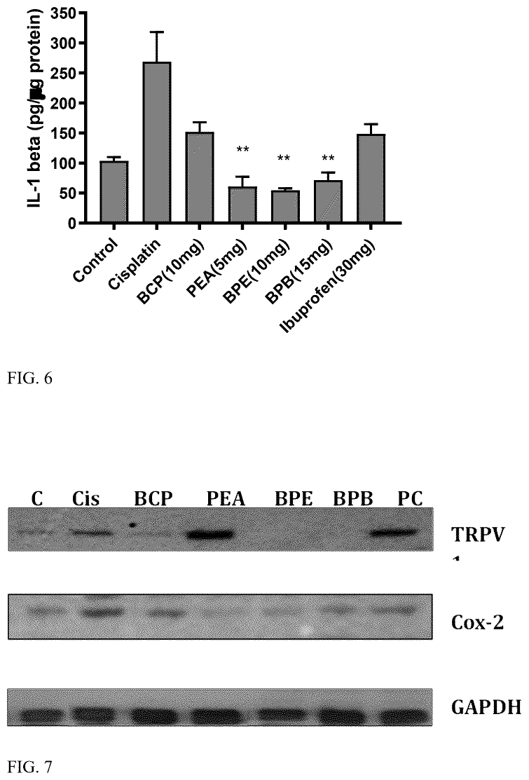

Cisplatin activates spinal microglia and upregulates the expression of inflammatory cytokines such as Interleukin-1β (IL-1β) and tumor necrosis factor-α (TNF-α). In the present study, these were estimated in the spinal fluids of the animals using ELISA kits as per the manufacturer's instruction (R&D Systems, Minneapolis, Minnesota, USA). From the results (Table 1), it is evident that 6% w/w Guineesine, β-Caryophyllene, each showed inhibition of Interleukin-1β (IL-1β) (FIG. 6) and tumor necrosis factor-α (TNF-α) (FIG. 5). However, the combination of Black pepper extract (6% w/w Guineesine) and β-Caryophyllene and Palmitoylethanolamide showed a synergistic inhibition (FIG. 5, FIG. 6).

### Estimation of TRPV and COX-2 Expression

The transient receptor potential vanilloid 1 (TRPV1), a nonselective cationic channel, regulates Ca²⁺ influx. TRPV1 seems to contribute to hypersensitivity to thermal, chemical, and mechanical stimuli associated with peripheral inflammation and neuronal damage. TRPV1 relies on its ability, following intense and prolonged activation, to induce neuronal desensitization. Pharmacological modulation of TRPV1 receptor is a promising way of producing analgesia in neuropathic pain models. Likewise, cyclooxygenase plays a key role in the nociceptive process, converting arachidonic acid to a variety of prostaglandins that can cause inflammation and pain. Cox-2 enzyme is expressed following immediate early gene upregulation at the site of injury in response to tissue damage and inflammatory stimuli. Cox-2 activity generates large amounts of prostaglandins (PGE2, PGI2) that activate signaling pathways in peripheral sensory neurons, increasing the sensitivity of these neurons to other painful stimuli such as heat, pressure, and protons. It plays a significant role in the central processing of the nociceptive or pain signals at the dorsal horn of the spinal cord and certain areas of the brain.

In the current study, TRPV1 and COX-2 expression was assessed in the spinal tissue lysate of the animals, using Western blotting. Briefly, proteins from the spinal tissue were separated in a 10% SDS-PAGE and transferred to PVDF membrane. The TRPV1 and COX-2 expressions were probed using specific monoclonal antibodies (ABclonal, Dusseldorf, Germany). GAPDH was used as housekeeping gene control. The expression of TRPV1 and COX-2 were normalized to that of GAPDH, and fold change was calculated w.r.t cisplatin group.

The results indicated that the combination of Black pepper extract (6% Guineesine) and β-Caryophyllene and Palmitoylethanolamide effectively reduced the expression of TRPV1 and COX-2 expression (FIG. 7, Table 2), thereby regulating Ca²⁺ influx and reducing inflammation.

TABLE 2
TRPV and COX-2 expression

| | | | | | | | Ibuprofen |
|---|---|---|---|---|---|---|---|
| | Control | Cisplatin | BCP(10 mg) | PEA(5 mg) | BPE(10 mg) | BPB(15 mg) | (30 mg) |
| TRPV1 | 0.3 | 1 | 0.1 | 2.8 | 0 | 0 | 1.8 |
| Cox 2 | 0.7 | 1 | 0.8 | 0.3 | 0.4 | 0.2 | 0.6 |

### Sensitivity to Pain Stimulus

Experimental models of pain sensitivity (nociception) include tests of response thresholds to high-intensity stimuli (acute pain tests) and of changes in spontaneous or evoked behavioral responses in animals. Pain cannot be directly measured in animals. Pain is inferred from “pain-like” behaviors, such as withdrawal from a nociceptive stimulus, which is the most commonly used method to quantify nociception in animal studies. If a stimulus is applied that does not normally evoke a withdrawal response, and the animal withdraws from the stimulus, the animal is considered to have allodynia, a type of neuropathic pain that makes people extremely sensitive to touch. Experimental models of pain sensitivity (nociception) include tests of response thresholds to high-intensity stimuli (acute pain tests) and of changes in spontaneous or evoked behavioral responses in animals.

### Tail-Flick Test

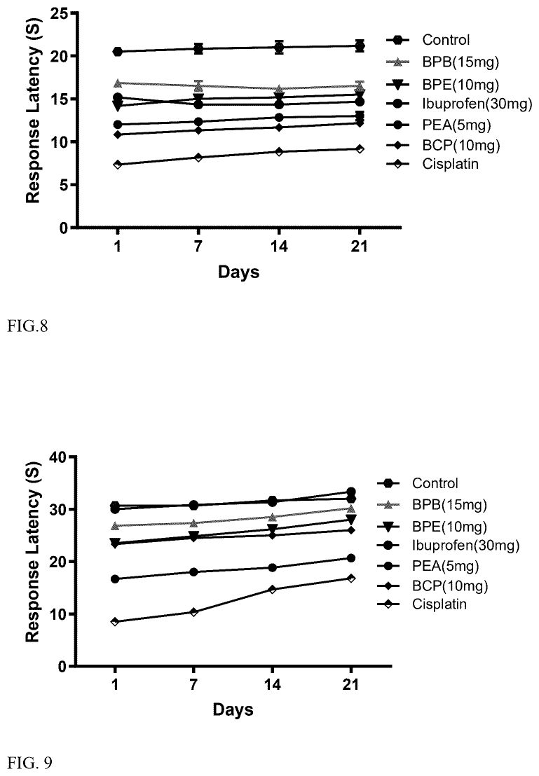

This is a test of acute nociception in which a high-intensity thermal stimulus is directed to the tail of a mouse or a rat. The time from onset of stimulation to a rapid flick/withdrawal of the tail from heat source is recorded. Thermal (radiant heat) sensation of animal tail part was assessed by observation of tail flick response as per D'Aemour and Smith method with minor modifications (Hargreaves et al. A new and sensitive method for measuring thermal nociception in cutaneous hyperalgesia. Pain 32 (1988) 77-88). The one-centimeter distance from the tail terminal region was located on the slit of the plantar device. Thermal sensitivity of mice tail was recorded as tail withdrawal latency. Brisk withdrawal of tail from the plantar device was considered as a progression of neuropathic pain. Cut-off stimuli, i.e., 15 s was maintained to avoid potential tissue damage. Mice were tested after 24 h of treatment injections for the drug-response evaluation. In Cisplatin group, the response latency time for tail flick test was significantly reduced (p<0.001), suggesting allodynia. Treatment with the combination was most effective in restoring the latency comparable to control animals (FIG. 8, Table 3)

### The Hot Plate Test

Animals are placed on the hot plate one at a time. Latency to respond to the heat stimulus is measured by the amount of time it takes for the animal to lick one of its paws. Animals were placed on a hot-plate apparatus. In order to measure thermal pain, the temperature of the hot plate surface was set to 52° C. The reaction latency as well as the time between when the animals were placed and the onset of paw-licking or jumping behaviors were measured in seconds(s). To minimize tissue damage, a cut-off time of 40 s was adopted (Moureq et al. Alleviation of cisplatin-induced neuropathic pain, neuronal apoptosis, and systemic inflammation in mice by rapamycin., Frontiers in Aging Neuroscience., 14, 2022). In Cisplatin group, the response latency time for hot plate test was significantly reduced (p<0.001), suggesting allodynia. Treatment with the combination was most effective in restoring the latency comparable to control animals (FIG. 9, Table 3).

TABLE 3
Animal Model to Study Pain

| | Tail Flick test | | Hot plate test | |
|---|---|---|---|---|
| Tukey's multiple | Mean | P | Mean | P |
| comparisons test | Diff. | value | Diff. | value |
| Cisplatin vs. | −3.125 | P < 0.001 | −12.13 | P < 0.001 |
| BCP (10 mg) | | | | |
| Cisplatin vs. | −4.167 | P < 0.001 | −5.958 | P < 0.01 |
| PEA (5 mg) | | | | |
| Cisplatin vs. | −6.583 | P < 0.001 | −13.04 | P < 0.001 |
| BPE (10 mg) | | | | |
| Cisplatin vs. | −8.125 | P < 0.001 | −15.63 | P < 0.001 |
| BPB (15 mg) | | | | |
| Cisplatin vs. | −6.25 | P < 0.001 | −18.79 | P < 0.001 |
| Ibuprofen (30 mg) | | | | |
| BCP (10 mg) vs. | −5 | P < 0.001 | −3.5 | NS |
| BPB (15 mg) | | | | |
| PEA (5 mg) vs. | −3.958 | P < 0.001 | −9.667 | P < 0.001 |
| BPB (15 mg) | | | | |
| BPE (10 mg) vs. | −1.542 | P < 0.01 | −2.583 | NS |
| BPB (15 mg) | | | | |
| BPB (15 mg) vs. | 1.875 | P < 0.01 | −3.167 | NS |
| Ibuprofen(30 mg) | | | | |

The results indicated that the combination of Black pepper extract (6% Guineesine) and β-Caryophyllene and Palmitoylethanolamide effectively reduced pain and modulating the associated biomarkers both in-vitro and in-vivo.

### Example 3: Ovalbumin Induced Respiratory Allergy

### Animal Groups for the Study

BALB/c mice (20-30 g body weight) were housed under standard laboratory conditions (temperature 23.5° C., humidity=58-64%), with 12 h light and 12 h dark cycle, fed with normal diet and water ad libitum. The protocol followed for animal experiment was approved by the institutional animal ethics committee (Table 3). The number of animals used in each group is 6. The composition was used in the ratio of 1:1:0.3 (BPE:BCP:PEA), however it is evident to the person skilled in the art that it is obvious to use any possible ratios to arrive at the expected results, the composition comprising 6% w/w Guineesine, β-Caryophyllene, and Palmitoylethanolamide in the ratios of 1-2:0.5-1:0.3-1 parts respectively, or preferably in the ratio of 1:0.5:0.5 parts respectively, or preferably in the ratio of 1:0.5:0.15 parts respectively, or preferably in the ratio of 1:1:0.33 parts respectively are also covered.

TABLE 4
Animal Groups for Allergy studies

| | |
|---|---|
| Group 1 | Normal Control |
| Group 2 | Disease Control |
| Group 3 | The Test Drug (β-Caryophyllene |
| | 10 mg/kg bodyweight) |
| Group 4 | The Test Drug (Palmitoylethanolamide |
| | 3 mg/kg bodyweight) |
| Group 5 | The Test Drug (6% w/w Guineesine |
| | (BPE)10 mg/kg bodyweight) |
| Group 6 | The Test Drug (Combination (BEP + |
| | BCP + PEA) 30 mg/kg |
| | bodyweight) (with actives alone 15 |
| | mg/kg bodyweight and with excipients |
| | it is 30 mg/kg bodyweight) |

Control animals were administered with 2% Gum acacia in PBS. All the animals except in Group I were actively sensitized by an intraperitoneal (i.p.) injection of OVA (20 μg/kg body weight) and Aluminum hydroxide (2 mg/kg body weight) in PBS on 0 and 14th day and were challenged with 1% OVA in PBS on 21^{st}, 22^{nd}, and 23^{rd} using Nebulizer for 1 hour. The treatment drugs and standard drug were administered orally for 5 days (18^{th} to 23^{rd} day).

On day 25, 48 h after final OVA challenge (saline challenge for normal control group), the animals were anesthetized for collection of blood and Bronchoalveolar Lavage (BAL) fluid for biochemical evaluation. After that the animals were sacrificed, their lungs and spleen were isolated, weighed and stored for further analysis.

### Organ Weight Determination

On the day of sacrifice, mice were anesthetized and their spleen and lungs was separated, moisture was removed by blotting with the filter paper, and the organ weight was taken using a digital weighing balance. It is widely known that lungs and spleen weight increase during allergy.

TABLE 5
Organ weight Determination

| | | | | | BCP | | PEA | | BPE | | BPB | |
|---|---|---|---|---|---|---|---|---|---|---|---|---|
| | Control | | OVA | | (10 mg/kg) | | (3 mg/kg) | | (10 mg/kg) | | (30 mg/kg) | |
| Lungs | 0.65 | 0.10 | 1.08 | 0.20** | 0.76 | 0.09** | 0.83 | 0.07* | 0.76 | 0.06** | 0.65 | 0.05*** |
| Spleen | 0.26 | 0.08 | 0.80 | 0.17** | 0.51 | 0.04** | 0.50 | 0.07** | 0.35 | 0.04** | 0.31 | 0.12*** |

^{#} Compared to control P < 0.001,
*compared to Ova,
*P < 0.01,
**P < 0.001,
***P, 0.0001

It is evident that 6% w/w Guineesine, β-Caryophyllene, and Palmitoylethanolamide (PEA) each showed decrease in organ weight. However, the combination of Black pepper extract (6% Guineesine) and β-Caryophyllene and Palmitoylethanolamide showed a synergistic decrease in organ weight compared OVA control and nearly as good as the healthy control (FIG. 10, Table 5).

### Evaluation of Nasal Symptoms

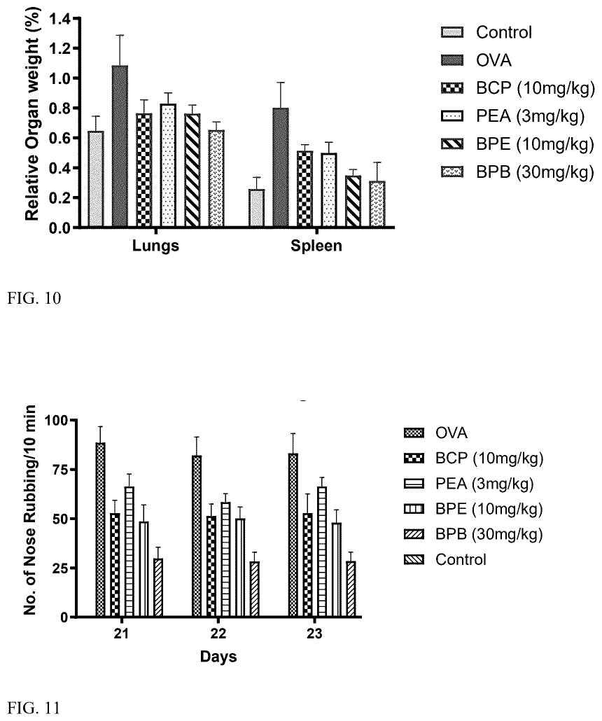

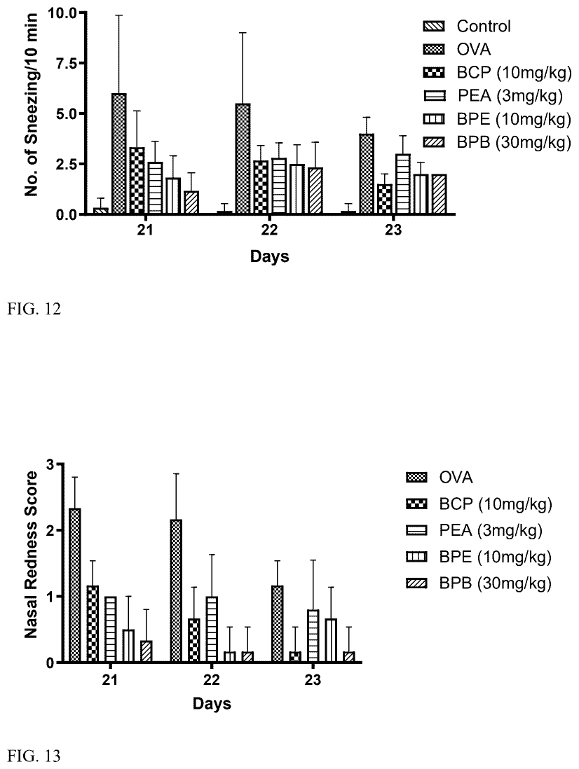

On day 21, 22 and 23 nasal symptoms were evaluated within a 10-minute period after the OVA challenge. The number of sneezes and nasal itching motions (nasal rubbing) (Chen et al. Apigenin Attenuates Allergic Responses of Ovalbumin-Induced Allergic Rhinitis Through Modulation of Th1/Th2 Responses in Experimental Mice. Dose Response. 2020 Mar. 3; 18 (1)) were recorded (FIG. 11, FIG. 12). Sneezing is characterized by an explosive expiration just after a deep inspiration and nose rubbing is characterized by an external perinasal scratch with either one or both forelimbs of animal. Another parameter that was evaluated was redness of the nose (FIG. 13) was by observing the intensity in redness of nostrils and scoring them as the following: 0—normal, 1—faint, 2—medium, and 3—dark. It is clear from the data (Tables 6-8, and FIG. 11-13) that comparatively it is the combination of Black pepper extract (6% w/w Guineesine, β-Caryophyllene and Palmitoylethanolamide showed a synergistic effect, individual components had little differential effect in each case, but collectively the combination showed synergistic effect in nasal symptoms.

TABLE 6
Nose rubbing

| Days | Control | | OVA | | BCP (10 mg/kg) | | PEA (3 mg/kg) | | BPE (10 mg/kg) | | BPB (30 mg/kg) | |
|---|---|---|---|---|---|---|---|---|---|---|---|---|
| 21.00 | 0.00 | 0.00 | 88.67 | 8.08^{#} | 52.83 | 6.52** | 66.40 | 6.28** | 48.67 | 8.34*** | 29.83 | 5.67*** |
| 22.00 | 0.17 | 0.37 | 82.17 | 9.32^{#} | 51.33 | 6.13*** | 58.40 | 4.32** | 50.17 | 5.76*** | 28.33 | 4.68*** |
| 23.00 | 0.17 | 0.37 | 83.17 | 10.07^{#} | 52.83 | 9.75*** | 66.40 | 4.54* | 48.00 | 6.51*** | 28.50 | 4.54*** |

^{#}Compared to control P < 0.001,
*compared to Ova,
*P < 0.01,
**P < 0.001,
***P, 0.0001

TABLE 7
Number of Sneezing

| | | | | | BCP | | PEA | | BPE | | BPB | |
|---|---|---|---|---|---|---|---|---|---|---|---|---|
| Days | Control | | OVA | | (10 mg/kg) | | (3 mg/kg) | | (10 mg/kg) | | (30 mg/kg) | |
| 21.00 | 0.33 | 0.47 | 6.00 | 3.87^{#} | 3.33 | 1.80 | 2.60 | 1.02 | 1.83 | 1.07* | 1.17 | 0.90* |
| 22.00 | 0.17 | 0.37 | 5.50 | 3.50^{#} | 2.67 | 0.75 | 2.80 | 0.75 | 2.50 | 0.96 | 2.33 | 1.25 |
| 23.00 | 0.17 | 0.37 | 4.00 | 0.82^{#} | 1.50 | 0.50*** | 3.00 | 0.89 | 2.00 | 0.58* | 2.00 | 0.00** |

^{#}Compared to control P < 0.001,
*compared to Ova,
*P < 0.01,
**P < 0.001,
***P, 0.0001

TABLE 8
Nasal Redness Score

| | | | BCP | | PEA | | BPE | | BPB | |
|---|---|---|---|---|---|---|---|---|---|---|
| Days | OVA | | (10 mg/kg) | | (3 mg/kg) | | (10 mg/kg) | | (30 mg/kg) | |
| 21.00 | 2.33 | 0.47^{#} | 1.17 | 0.37*** | 1.00 | 0.00*** | 0.50 | 0.50*** | 0.33 | 0.47*** |
| 22.00 | 2.17 | 0.69^{#} | 0.67 | 0.47*** | 1.00 | 0.63* | 0.17 | 0.37*** | 0.17 | 0.37*** |
| 23.00 | 1.17 | 0.37^{#} | 0.17 | 0.37*** | 0.80 | 0.75 | 0.67 | 0.47 | 0.17 | 0.37** |

^{#}Compared to control P < 0.001,
*compared to Ova,
*P < 0.01,
**P < 0.001,
***P, 0.0001

### Evaluation of Bronchoalveolar Lavage Fluid (BALF) Cell Count

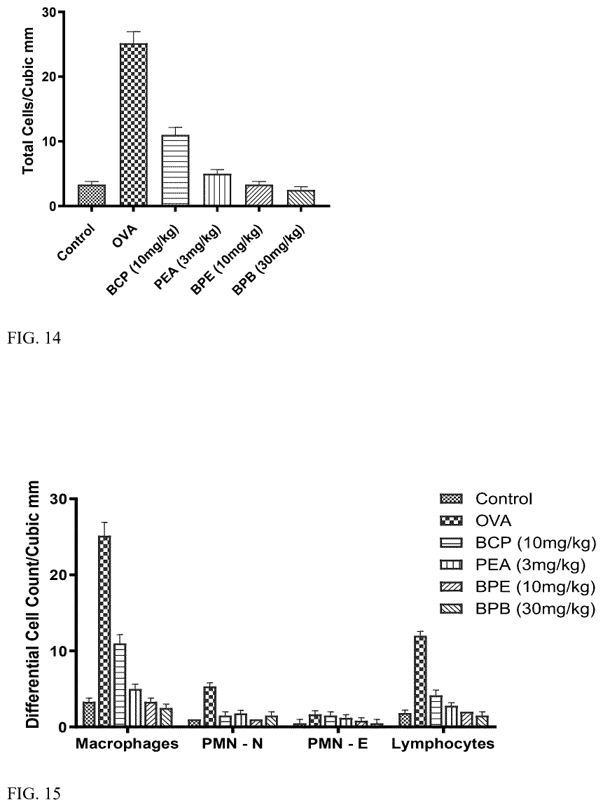

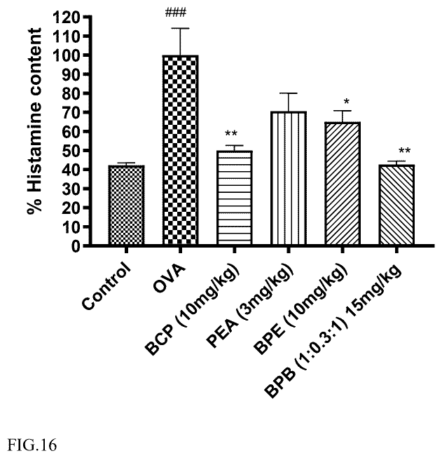

On the day of sacrifice mice were anesthetized, the lungs were gently lavaged three times via the tracheal tube with a volume of 0.7 ml phosphate buffered saline (PBS). BALF was immediately centrifuged at 1500 rpm for 5 min, and the collected supernatant was stored at −70° C. until further analysis. The cell pellet was analysed to determine the total cell count of BALF (Table 9, FIG. 14) and the count of Macrophages, neutrophil, eosinophil, and lymphocyte (Table 10, FIG. 15) counts using a hematology analyzer in BALF fluids. The levels of Macrophages, Neutrophil, Eosinophil, and Lymphocyte are elevated during allergy, and the individual components had effects (Table 10, FIG. 15), but not the extent the combination showed synergistic effect (Table 10, FIG. 16).

TABLE 9
Cellular Count

| | | | | BCP | | PEA | | BPE | | BPB | |
|---|---|---|---|---|---|---|---|---|---|---|---|
| Control | | OVA | | (10 mg/kg) | | (3 mg/kg) | | (10 mg/kg) | | (30 mg/kg) | |
| 6.93 | 0.69 | 44.17 | 1.57 | 18.17 | 0.69*** | 10.80 | 0.75*** | 7.33 | 0.47*** | 6.00 | 0.82*** |

TABLE 10
Differential Count

| | | | | | BCP | | PEA | | BPE | | BPB | |
|---|---|---|---|---|---|---|---|---|---|---|---|---|
| | Control | | OVA | | (10 mg/kg) | | (3 mg/kg) | | (10 mg/kg) | | (30 mg/kg) | |
| Macrophages | 3.33 | 0.47 | 25.17 | 1.77^{##} | 11.00 | 1.15*** | 5.00 | 0.63*** | 3.33 | 0.47*** | 2.50 | 0.50*** |
| PMN - N | 1.00 | 0.00 | 5.33 | 0.47^{##} | 1.50 | 0.50*** | 1.80 | 0.40*** | 1.00 | 0.00*** | 1.50 | 0.50*** |
| PMN - E | 0.50 | 0.50 | 1.67 | 0.47^{#} | 1.50 | 0.50 | 1.20 | 0.40 | 0.83 | 0.37** | 0.50 | 0.50*** |
| Lymphocytes | 1.83 | 0.37 | 12.00 | 0.58^{##} | 4.17 | 0.69*** | 2.80 | 0.40*** | 2.00 | 0.00*** | 1.50 | 0.50*** |

^{#}Compared to control P < 0.01,
^{##}p < 0.001,
*compared to Ova,
*P < 0.01,
**P < 0.001,
***P, 0.0001

### Anti-Histamine Effects in BALF and Mast Cells

Human mast cells play a significant role in human asthma. Histamine in bronchoalveolar lavage fluid (BALF) used as an indicator of pulmonary mast cell activation. Histamine levels in BALF was measured using the alkaline condensation reaction of o-phthaldialdehyde (OPT) with histamine as described by briefly, 100 μl of samples were incubated in the presence of NaOH (2 N), and OPT (0.625 mg/ml) for exactly 10 min. The reaction was stopped by the addition of 50 μl of HCL (IN), and the fluorescence was measured on BMG Fluostar Optima microplate reader at Ex/Em: 320/460 nm. Lower the levels of histamine indicative of no allergy or lack of activation of mast cells. It is evident that the combination had a better synergistic effect compared to the individual components (FIG. 16). To corroborate the finding that antihistamine effect is through inhibition of mast cell activation (refer FIGS. 4A-4D), it is clear that each of the components had an effect and the combination showed a comprehensive effect.

### Example 4: Formulations

Tables 11-23 provide illustrative examples of nutraceutical formulations

TABLE 11
Tablet

Active Ingredients
4-10% Guineesine (BPE)
β-Caryophyllene (BCP)
Excipients
Microcrystalline cellulose, Colloidal silicon dioxide, Magnesium stearate,
BioPerine ®, Polyvinylpyrrolidone/starch/Hydroxy propyl methyl
cellulose, Hydroxy propyl cellulose

TABLE 12
Tablet

Active Ingredients
4-10% Guineesine (BPE)
β-Caryophyllene (BCP)
Palmitoylethanolamide (PEA)
Excipients
Microcrystalline cellulose, BioPerine ®

TABLE 13
Tablet

Active Ingredients
4-10% Guineesine (BPE)
β-Caryophyllene (BCP)
Palmitoylethanolamide (PEA)
Excipients
Microcrystalline cellulose

TABLE 14
Capsule

Active Ingredients
4-10% Guineesine (BPE)
β-Caryophyllene (BCP)
Excipients
Microcrystalline cellulose, Colloidal silicon dioxide, Magnesium stearate,
BioPerine ®, Polyvinylpyrrolidone/starch/Hydroxy propyl methyl
cellulose, Hydroxy propyl cellulose

TABLE 15
Capsule

Active Ingredients
4-10% Guineesine (BPE)
β-Caryophyllene (BCP)
Palmitoylethanolamide (PEA)
Excipients
Microcrystalline cellulose, Colloidal silicon dioxide, Magnesium stearate,
BioPerine ®, Polyvinylpyrrolidone/starch/Hydroxy propyl methyl
cellulose, Hydroxy propyl cellulose

TABLE 16
Capsule

Active Ingredients
4-10% Guineesine (BPE)
β-Caryophyllene (BCP)
Palmitoylethanolamide (PEA)
Excipients
Microcrystalline cellulose, Colloidal silicon dioxide, Magnesium stearate,
Polyvinylpyrrolidone/starch/Hydroxy propyl methyl cellulose, Hydroxy
propyl cellulose

TABLE 17
Powder

Active Ingredients
4-10% Guineesine (BPE)
β-Caryophyllene (BCP)
Excipients
BioPerine ®,

TABLE 18
Gummy Formulation

Active Ingredients
4-10% Guineesine (BPE)
β-Caryophyllene (BCP)
Excipients
BioPerine ®, Gelatin (270 Bloom Mesh 10), Refined Sugar, Glucose Corn
Syrup, Citric Acid, Lactic Acid, Water, Natural Mango Flavor M38630,
Tartaric Acid, Refined Sugar

TABLE 19
Gummy Formulation

Active Ingredients
4-10% Guineesine (BPE)
β-Caryophyllene (BCP)
Palmitoylethanolamide (PEA)
Excipients
BioPerine ®, Gelatin (270 Bloom Mesh 10), Refined Sugar, Glucose Corn
Syrup, Citric Acid, Lactic Acid, Water, Natural Mango Flavor M38630,
Tartaric Acid, Refined Sugar

TABLE 20
Gummy Formulation

Active Ingredients
4-10% Guineesine (BPE)
β-Caryophyllene (BCP)
Palmitoylethanolamide (PEA)
Excipients
Gelatin (270 Bloom Mesh 10), Refined Sugar, Glucose Corn Syrup, Citric
Acid, Lactic Acid, Water, Natural Mango Flavor M38630, Tartaric Acid,
Refined Sugar

TABLE 21
Candy Formulation

Active Ingredients
4-10% Guineesine (BPE)
β-Caryophyllene (BCP)
Excipients
BioPerine ®, Sucrose, Liquid Glucose, Flavoring agent, Menthol,
Acidulants (Citric acid/Tartaric Acid/Maleic Acid), Purified water

TABLE 22
Candy Formulation

Active Ingredients
4-10% Guineesine (BPE)
β-Caryophyllene (BCP)
Palmitoylethanolamide (PEA)
Excipients
BioPerine ®, Sucrose, Liquid Glucose, Flavoring agent, Menthol,
Acidulants (Citric acid/Tartaric Acid/Maleic Acid), Purified water

TABLE 23
Candy Formulation

Active Ingredients
4-10% Guineesine (BPE)
β-Caryophyllene (BCP)
Palmitoylethanolamide (PEA)
Excipients
Sucrose, Liquid Glucose, Flavoring agent, Menthol, Acidulants (Citric
acid/Tartaric Acid/Maleic Acid), Purified water

The above formulations are merely illustrative examples, any formulation containing the above active ingredient intended for the said purpose will be considered equivalent.

To summarize, Itch and pain are closely related with similarities and differences, largely by modulating histamine levels through activation of mast cells. The composition comprising 4-10% Guineesine and β-Caryophyllene have shown effective management of pain (mast cell activation) and allergy (BALF) clearly suggesting a common pathway through inhibiting histamine levels by moderating mast cell activation. Also, the composition has shown synergistic effect in neuropathic and inflammatory pain.

Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention and is to be interpreted only in conjunction with the appended claims.

We claim:

1. A composition comprising 4-10% w/w Guineesine and β-Caryophyllene.

2. The composition as in claim 1, further comprises Palmitoylethanolamide.

3. (canceled)

4. The composition as in claim 1, wherein the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

5. A method of alleviating pain in a subject, comprising a step of administering to a subject in need thereof, an effective dose of a composition comprising 4-10% w/w Guineesine and β-Caryophyllene to alleviate pain in the subject.

6. The method as in claim 5, wherein the composition further comprises Palmitoylethanolamide.

7. The method as in claim 5, wherein the composition can be used individually or in combination.

8. The method as in claim 5, wherein effective dose in vitro is in the range 0.1-20 μg/mL and the effective dose in vivo is in the range 1-30 mg/Kg.

9. The method as in claim 5, wherein the composition alleviates the pain selected from the group consisting of nociception, inflammation, and neuropathic.

10. The method as claimed in claim 5, wherein the manifestation of pain results in conditions selected from hyperalgesia, allodynia.

11. The method as in claim 5, wherein the alleviation of pain is mediated by, decrease of COX-2 expression, inhibition of intracellular calcium release by modulating the expression of TRPV receptors, inhibition of nitric oxide, inhibition of IL-6, and inhibition of histamine release from mast cell degranulation.

12. The method as in claim 5, wherein the subject is selected from the group consisting of a mammalian cell or mammal.

13. The method as in claim 5, wherein the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.

14. A method of alleviating allergy in a subject, comprising a step of administering to a subject in need thereof, an effective dose of a composition comprising 4-10% w/w Guineesine and β-Caryophyllene to alleviate allergy in the subject.

15. The method as in claim 14, the composition further comprises Palmitoylethanolamide.

16. The method as claimed in claim 14, wherein the composition can be used individually or in combination.

17. The method as in claim 14, wherein the effective dose in vitro is in the range 0.1-20 μg/mL and the effective dose in vivo is in the range 1-30 mg/Kg.

18. The method as in claim 14, wherein the composition alleviates allergy mediated by allergens selected from the group consisting of eggs, milk, mustard, peanuts, crustaceans and mollusks, fish, sesame seeds, soy, sulphites, tree nuts, wheat and triticale, histamine rich foods, ovalbumin, environmental pollutant and chemicals, antibiotics and drugs.

19. The method as in claim 14, wherein the alleviation of allergy is mediated by inhibition of histamine release from mast cells reduction in nasal symptoms and reduction of bronchoalveolar lavage fluid (BALF) cell count.

20. The method as in claim 14, wherein the subject is selected from the group consisting of a mammalian cell or mammal.

21. The method as in claim 15, wherein the composition is formulated with pharmaceutically/nutraceutically acceptable excipients, adjuvants, diluents or carriers, and administered orally in the form of tablets, capsules, syrups, gummies, powders, suspensions, emulsions, chewables, candies, and eatables.
